(19) United States
(12) Patent Application Publication
(10) Pub. No.: US 2008/0112933 A1
(43) Pub. Date: May 15, 2008

(54) Methods and Compositions for Increasing Stem Cell Homing Using Gas Activators

(76) Inventors: David T. Scadden, Weston, MA (US); Henry M. Kronenberg, Belmont, MA (US); Gregor Adams, Boston, MA (US)

Correspondence Address: EWARDS ANGELL PALMER & DODGE LLP, P.O. BOX 55874, BOSTON, MA, 02205, US

(21) Appl. No.: 11/667,329

(22) PCT Filed: Nov. 7, 2005
(86) PCT No.: PCT/US05/40416

§ 371 Date: Nov. 2, 2007

Related U.S. Application Data
(60) Provisional application No. 60/625,914, filed on Nov. 8, 2004.

(51) Int. Cl. A61K 35/14; C12N 5/08

(52) U.S. Cl. 424/93.7; 435/372

(57) ABSTRACT

The present invention provides methods for increasing engraftment of stem cells in a subject by treating the cells with a Gαs activator. The invention further provides methods for identifying Gαs activators for use in increasing engraftment of stem cells in a subject.

### CROSS-REFERENCE TO RELATED APPLICATIONS

This application claims the benefit of U.S. Provisional Application No. 60/625,914, which is hereby incorporated by reference in its entirety. Each of the applications and patents cited in this text, as well as documents or references cited in each of the applications and patents (including during the prosecution of each issued patent; “application cited document”) and each of the PCT and foreign applications and patents, and each of the documents cited or referenced in each of the application cited documents, are hereby expressly incorporated herein by reference, and may be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference. Documents incorporated by reference into this text or any teaching therein can be used in the practice of this invention.

### BACKGROUND OF THE INVENTION

During development, hematopoietic stem cells (HSCs) translocate from the fetal liver to the bone marrow, which remains the site of hematopoiesis throughout adulthood. The exogenous signals that specifically direct hematopoietic stem cells to the bone marrow have been thought to include stimulation of the chemokine receptor CXCR4 by its cognate ligand stromal derived factor-1α (SDF-1α or CXCL12). Studies with mice engineered to be deficient in either CXCR4 or SDF-1α have demonstrated a failure of stem cell translocation to the bone marrow (Nagasawa et al, 1996; Ma et al, 1998). However, experiments in which CXCR4^{-/-} fetal liver hematopoietic cells were transplanted into wild-type hosts demonstrated efficient engraftment of the HSCs in the bone marrow. Only a subset of the hematopoietic progenitor cells failed to engraft the bone marrow (Kawabata et al, 1999; Ma et al, 1999), suggesting that CXCR4 is not the sole basis for bone marrow localized hematopoiesis. In addition, treatment of HSCs with inhibitors of Gαi-coupled signaling, which blocks HSC transmigration towards SDF-1α in vitro, does not affect bone marrow homing and engraftment in vivo (Wiesmann & Spangrude, 1999; Kollet et al, 2001). Therefore other mechanisms for the homing of HSCs to the bone marrow may exist.

Bone marrow transplants are often used to treat patients diagnosed with leukemia, aplastic anemia, lymphomas such as Hodgkin's disease, multiple myeloma, immune deficiency disorders and some solid tumors such as breast and ovarian cancer. HSC homing to the bone marrow is critical for the success of bone marrow transplantation. Historically, bone marrow transplants have been performed using allogenic bone marrow. More recently, transplants have been performed using hematopoietic stem cells isolated from peripheral blood, as well as from umbilical cord blood, which carries a lower risk to the recipient of graft-versus-host disease.

However, umbilical cord blood often does not contain sufficient quantities of stem cells to sufficiently repopulate the bone marrow of adult recipients, and it may also be difficult to isolate sufficient numbers of stem cells from peripheral blood. Accordingly, there exists a need in the art for methods that can increase the efficiency of stem cell homing to the bone marrow, in order to broaden the applicability and increase the success of stem cell and umbilical cord blood transplants.

### SUMMARY OF THE INVENTION

The present invention is based, at least in part, on the discovery that the alpha subunit of the stimulatory G-protein (“Gαs”) is required for bone marrow localization of stem cells. The present invention is further based on the discovery that modulation of Gαs can increase or decrease stem cell localization to the bone marrow. Accordingly, the present invention provides methods for increasing stem cell engraftment which include treating the cells with Gαs activators. The present invention also provides methods for identifying Gαs activators for use in increasing stem cell engraftment.

In one aspect, blood cells are contacted with a Gαs activating agent and administered to a subject in need thereof.

In one embodiment, the invention provides a method for increasing stem cell engraftment in a subject in need thereof (e.g., a human) comprising contacting blood cells (e.g., bone marrow cells, hematopoietic stem or progenitor cells, and/or umbilical cord blood cells) to be transplanted into the subject with a Gαs activating agent (e.g., cholera toxin) and administering the cells to the subject, thereby increasing stem cell engraftment in the subject.

In a specific embodiment, the blood cells engraft into the bone marrow of the subject.

In another embodiment, the invention provides a method for increasing stem cell mobilization to the bone marrow of a subject in need thereof comprising contacting blood cells to be transplanted into the subject with a Gαs activating agent and administering the cells to the subject, thereby increasing stem cell mobilization to the bone marrow of the subject.

In another aspect, stem and/or progenitor cells are contacted with a Gαs activating agent and administered to a subject in need thereof.

In one embodiment, the invention provides a method for increasing stem cell engraftment in a subject in need thereof (e.g., a human) comprising contacting stem and/or progenitor cells to be transplanted into the subject with a Gαs activating agent (e.g., cholera toxin) and administering the cells to the subject, thereby increasing stem and/or progenitor cell engraftment in the subject.

In a specific embodiment, the stem and/or progenitor cells engraft into the bone marrow of the subject.

In another embodiment, the invention provides a method for increasing stem cell mobilization to the bone marrow of a subject in need thereof comprising contacting stem and/or progenitor cells to be transplanted into the subject with a Gαs activating agent and administering the cells to the subject, thereby increasing stem and/or progenitor cell mobilization to the bone marrow of the subject.

In one embodiment, the methods of the invention include substantially removing the Gαs activating agent from the cells prior to administering the cells to the subject, e.g., by washing the cells.

In yet another embodiment, the methods further comprises treating subjects with an amount of radiation or chemotherapy sufficient to ablate the bone marrow in the subjects prior to administration of the cells.

In yet another embodiment, the subjects are suffering from a disorder selected from the group consisting of leukemia, aplastic anemia, lymphoma (Hodgkin's disease or Non-Hodgkin's lymphoma), multiple myeloma, an immune disorder (severe combined immune deficiency syndrome or lupus), myelodysplasia, thalassemaia, and sickle-cell disease, Wiskott-Aldrich syndrome, and solid tumors (breast cancer, ovarian cancer, brain cancer, prostate cancer, lung cancer, colon cancer, skin cancer, liver cancer, or pancreatic cancer).

In another aspect, the invention provides kits containing Gαs activating agents.

In one embodiment, the invention provides a kit for increasing stem and/or progenitor cell engraftment in a subject in need thereof comprising a Gαs activating agent and instructions for using a Gαs activating agent to increase stem and/or progenitor cell engraftment in the subject in accordance with the methods herein.

In another embodiment, the invention provides a kit for increasing stem and/or progenitor cell mobilization to the bone marrow of a subject in need thereof comprising a Gαs activating agent and instructions for using a Gαs activating agent to increase stem and/or progenitor cell mobilization to the bone marrow of the subject in accordance with the methods herein.

In another aspect, the invention provides a method for identifying an agent capable of increasing stem and/or progenitor cell engraftment comprising:
a) providing a first portion of cells which express Gαs;
b) providing a second portion of cells which do not express Gαs;
c) contacting the first and second portions of cells with a test compound; and
d) detecting cAMP expression in both the first and second portions of cells,

wherein a test compound which increases cAMP expression from the first portion of cells but not the second portion of cells is identified as an agent capable of increasing stem and/or progenitor cell engraftment. In a specific embodiment, the cells are bone marrow mononuclear cells or bone marrow lin⁻ cells. In a further specific embodiment, the method comprises:
e) providing a third portion of cells which are hematopoietic progentor cells and a fourth portion of cells which are hematopoietic progenitor cells;
f) contacting the third portion of cells with the test compound identified as an agent capable of increasing stem and/or progenitor cell engraftment;
g) administering the third portion of cells to a test subject;
h) administering the fourth portion of cells to a test subject; and
i) detecting engraftment of the third and fourth portion of cells in the bone marrow of the test subjects,

wherein a test compound which increases the number of engrafted cells from the third portion of cells, as compared to the fourth portion of cells, is confirmed as agent capable of increasing stem cell engraftment. In a preferred embodiment, the third and fourth portions of cells are bone marrow lin⁻ cells. In a further preferred embodiment, the subject is a mouse. In still a further preferred embodiment, steps (h) and (j) are performed eight weeks apart.

Other features and advantages of the invention will be apparent from the following detailed description and claims.

### BRIEF DESCRIPTION OF THE DRAWINGS

FIG. 1 depicts cross sections of E17.5 humerus (left) and tibia (right) bones from wild-type/Gαs^{-/-} chimeric mice at embryonic day 17.5 (E17.5). Gαs^{-/-} cells are marked by neo mRNA expression. No Gαs^{-/-} cells are seen in the bone marrow.

FIG. 2 depicts the results of colony-forming-unit assays using wild-type and Gαs^{-/-} cells. Gαs^{-/-} cells are able to differentiate into hematopoietic cells as well as wild-type cells.

FIG. 3 depicts the detection of Gαs activity in bone marrow mononuclear cells (MNCs) treated with or without suramin, an inhibitor of Gαs activity. Gαs^{-/-} activity was measured by detecting cAMP production.

FIG. 4 depicts the results of a long-term culture-initiating cell assay (LTC-IC) in bone marrow MNCs treated with or without suramin, an inhibitor of Gαs activity. Inhibition of Gαs activity does not impair the ability of the cells to differentiate into hematopoietic cells.

FIG. 5 depicts the results of an in vitro SDF-1α a transmigration assay towards using bone marrow lin⁻ cells treated with suramin or pertussis toxin.

FIG. 6 depicts the results of a bone marrow homing assay using lin⁻ cells treated with suramin or pertussis toxin.

FIG. 7 depicts the results of a lymph node homing assay using lymph node lymphocytes treated with or without suramin or pertussis toxin.

FIG. 8 depicts the results of a competitive repopulation assay using bone marrow MNCs treated with or without suramin.

FIG. 9 depicts the results of a homing and engraftment assay following treatment of hematopoietic progenitor cells with cholera toxin, a stimulator of Gαs activity.

FIG. 10 depicts the results of a competitive repopulation assay following treatment of hematopoietic progenitor cells with cholera toxin.

FIGS. 11A, 11B, and 11C are graphs showing that Gsα does not affect hematopoietic differentiation capabilities of hematopoietic stem cells. FIG. 11A is a graph showing the results of a long-term culture-initiating cell (LTC-IC) assay bone marrow mononuclear cells obtained from mice with the conditional knockout of Gₛα, or Gₛa^{+/+} Mx1-Cre mice (wild-type) showed no difference in their LTC-IC frequency. FIG. 11B showing the number of number of Gₛα knockout cells localizing to the bone marrow and spleen as calculated by flow cytometry. FIG. 11C is a graph showing the in vivo bone marrow reconstituting ability of wild-type versus knockout cells as calculated by flow cytometry.

### DETAILED DESCRIPTION OF THE INVENTION

### I. Definitions

As used herein, “blood cells” are any population of cells derived from a hematopoietic source including bone marrow cells, hematopoietic stem or progenitor cells and/or umbilical cord blood cells.

“Stem cells” are immature cells having the capacity to self-renew and to differentiate into the more mature cells. Progenitor cells also have the capacity to self-renew and to differentiate into more mature cells, but are committed to a lineage (e.g., hematopoietic progenitors are committed to the blood lineage), whereas stem cells are not necessarily so limited. For the purposes of this disclosure, progenitor cells can be interchangeably described as “stem cells” throughout the specification.

The term “engraft” refers to the ability of a cell to contact and integrate into a tissue, such as the bone marrow.

“Homing” refers to the ability of migratory stem or progenitor cells to localize and engraft into a particular tissue, such as the bone marrow.

A “Gαs activating agent” includes any agent capable of activating the alpha subunit of the stimulatory G-protein (“Gαs”) or variants of Gαs.

The term “obtaining” as in “obtaining the agent that activates Gαs” is intended to include purchasing, synthesizing or otherwise acquiring the agent (or indicated substance or material).

As used herein, a “subject” is a human, non-human primate, cow, horse, pig, sheep, goat, dog, cat or rodent.

Other definitions appear in context throughout this disclosure.

### II. Methods of the Invention

It has been discovered according to some aspects of the invention that activation of Gαs increases the capacity of blood cells to mobilize and engraft into host tissues, including the bone marrow. This effect can be mediated by activation of Gαs by various activating agents, such as cholera toxin. This represents an unexpected discovery with important clinical implications for the field of blood cell transplantation.

Expanding the number of bone marrow derived progenitor cells is a long-sought solution to the inadequate number of stem and progenitor cells available for transplantation in hematologic and oncologic disease. Currently approximately 25% of autologous donor transplants are prohibited for lack of sufficient progenitor cells. In addition, less than 25% of patients in need of allogeneic transplant can find a histocompatible donor. Umbilical cord blood banks currently exist and cover the broad racial make-up of the general population, but are currently restricted in use to children due to inadequate progenitor cell numbers in the specimens. Methods of the invention maximize the potential of harvested blood cell samples by increasing the homing capacity of stem and progenitor cells. Methods of the invention thereby increase the efficiency of transplantation and potentially reduce the time and discomfort associated with bone marrow/peripheral progenitor cell harvesting.

Blood cells of the invention are any population of cells derived from a hematopoietic source suitable for use in transplantation or further purification prior to transplantation, including bone marrow cells, hematopoietic stem or progenitor cells and/or umbilical cord blood cells. It is known in the art that hematopoietic progenitor cells can include CD34⁺ cells. CD34⁺ cells are immature cells present in the hematopoietic sources described below, express the CD34 cell surface marker, and are believed to include a subpopulation of cells with the “progenitor cell” properties defined above. In a specific embodiment, the blood cells either contain or are a purified population of bone marrow mononuclear cells or bone marrow lin⁻ cells. Fractions of lin⁻ cells are known in the art to contain hematopoietic stem and progenitor cells. Blood cells of the invention can include the progeny of hematopoietic stem and progenitor cells, including granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and monocytes (e.g., monocytes, macrophages).

The blood cells of the invention can be obtained from hematopoietic sources, such as an organ of the body containing cells of hematopoietic origin. Such sources include unfractionated bone marrow, umbilical cord, peripheral blood, liver, thymus, lymph and spleen. It will be apparent to those of ordinary skill in the art that all of the aforementioned crude or unfractionated sources can be enriched for cells having hematopoietic progenitor cell characteristics in a number of ways. For example, the sources can be depleted of the more differentiated progeny. The more mature, differentiated cells can be selected against, via cell surface molecules they express. Additionally, the sources can be fractionated selecting for CD34⁺ cells. As mentioned earlier, CD34⁺ cells are thought in the art to include a subpopulation of cells capable of self-renewal and pluripotentiality. Such selection can be accomplished using, for example, commercially available magnetic anti-CD34 beads (Dynal, Lake Success, N.Y.). Unfractionated sources can be obtained directly from a donor or retrieved from cryopreservative storage.

In specific embodiments of the invention, hematopoietic stem and progenitor cells may be harvested from a hematopoietic source. “Harvesting” hematopoietic progenitor cells is defined as the dislodging or separation of cells from the matrix. This can be accomplished using a number of methods, such as enzymatic, non-enzymatic, centrifugal, electrical, or size-based methods, or preferably, by flushing the cells using media (e.g. media in which the cells are incubated). The cells can be further collected, separated, and further expanded generating even larger populations of differentiated progeny.

The blood cells of the invention can be obtained from differentiated pluripotent or multipotent stem cells. In other embodiments of the invention, pluripotent or multipotent stem cells can be treated with a Gαs activating agent and administered to the subject without additional differentiation steps.

Pluripotent stem cells of the present invention include embryonic stem cells. The quintessential stem cell is the embryonic stem (ES) cell, as it has unlimited self-renewal and pluripotent differentiation potential (Thomson, J. et al. 1995; Thomson, J. A. et al. 1998; Shamblott, M. et al. 1998; Williams, R. L. et al. 1988; Orkin, S. 1998; Reubinoff, B. E., et al. 2000). These cells are derived from the inner cell mass (ICM) of the pre-implantation blastocyst (Thomson, J. et al. 1995; Thomson, J. A. et al. 1998; Martin, G. R. 1981), or can be derived from the primordial germ cells from a post-implantation embryo (embryonal germ cells or EG cells). ES and/or EG cells have been derived from multiple species, including mouse, rat, rabbit, sheep, goat, pig and more recently from human and human and non-human primates (U.S. Pat. Nos. 5,843,780 and 6,200,806).

When introduced into mouse blastocysts, ES cells can contribute to all tissues of the mouse (animal) (Orkin, S. 1998). Murine ES cells are therefore known to be pluripotent. When transplanted in post-natal animals, ES and EG cells generate teratomas, which again demonstrates their multipotency. ES (and EG) cells can be identified by positive staining with the antibodies to stage-specific embryonic antigens (SSEA) 1 and 4.

Pluripotent embryonic stem cells are well known in the art. For example, U.S. Pat. Nos. 6,200,806 and 5,843,780 refer to primate, including human, embryonic stem cells that are stated to proliferate in an in vitro culture for over one year, maintain a karyotype in which the chromosomes are euploid and not altered through prolonged culture, maintain the potential to differentiate to derivatives of endoderm, mesoderm, and ectoderm tissues throughout the culture, and are inhibited from differentiation when cultured on a fibroblast feeder layer.

U.S. Patent Applications Nos. 20010024825 and 20030008392 describe human embryonic stem cells that are stated to proliferate in an in vitro culture for over one year, maintain a karyotype in which all the chromosomes characteristic of the human species are present and not altered through prolonged culture, maintain the potential to differentiate to derivatives of endoderm, mesoderm, and ectoderm tissues throughout the culture, and are inhibited from differentiation when cultured on a fibroblast feeder layer. U.S. Patent Application No. 20030113910 describes pluripotent non-embryonic stem cells, which are stated to be capable of proliferating in an in vitro culture for more than one year; maintain a karyotype in which the cells are euploid and are not altered through culture; maintain the potential to differentiate into cell types derived from the endoderm, mesoderm and ectoderm lineages throughout the culture, and are inhibited from differentiation when cultured on fibroblast feeder layers.

U.S. Patent Application No. 20030073234 describes a clonal human embryonic stem cell line stated to be capable of sustaining a normal embryonic stem cell phenotype following at least eight months of in vitro culturing.

U.S. Pat. No. 6,090,625 and U.S. Patent Application No. 20030166272 describe an undifferentiated cell that is stated to be pluripotent.

An undifferentiated human embryonic stem cell is described in U.S. Patent Application No. 20020160509. The cell is stated to be immunoreactive with markers for human pluripotent stem cells including SSEA-4, GCTM-2 antigen, and TRA 1-60, and also expresses Oct-4.

U.S. Patent Application No. 20020081724 describes what are stated to be embryonic stem cell derived cell cultures, isolated by disaggregation of embryonic stem cells and embryoid bodies (EBs).

Other kinds of pluripotent stem cells are also well known in the art. U.S. Pat. No. 5,827,735 describes mesenchymal stem cells that are stated to be pluripotent. The mesenchymal stem cells can form fibroblastic cells as well as multinucleated structures that spontaneously contract when induced to differentiate.

An embryonic-like stem cell derived from non-embryonic or postnatal animal cells or tissues, and stated to be a pluripotent (e.g., can give rise to cells of endodermal, ectodermal and mesodermal lineages), capable of self-renewal and differentiation into cells of endodermal, ectodermal and mesodermal lineages, is described in U.S. Patent Application No. 20030161817.

U.S. Pat. No. 5,914,268 describes a pluripotent cell population that is stated to be pluripotent for development into hematopoietic cells, progenitors and progeny thereof. The pluripotent cell population is derived by culturing an embryonic stem cell population to obtain an embryoid body cell population, which is then followed by culturing said embryoid body cell population under conditions effective to produce said pluripotent cell population. The culturing conditions comprise an embryonic blast cell medium.

U.S. Patent Application No. 20030157078 refers to an isolated pluripotent pre-mesenchymal, pre-hematopoietic progenitor stem cell. Such cells are stated to have the potential to differentiate into both mesenchymal and hematopoietic phenotypes, as determined by a proliferative response to inductive growth factors and cytokines, and by their morphologic and cytochemical features.

U.S. Patent Application No. 20030161817 refers to cultured isolates comprising stem cells isolated from an umbilical cord matrix source of stem cells, other than cord blood, the isolate comprising totipotent immortal stem cells. These cell isolates are stated to be capable of proliferation in an in vitro culture for over one year, can maintain a karyotype in which all the chromosomes characteristic of the human are present and not noticeably altered through prolonged culture; and maintain the potential to differentiate into derivatives of endoderm, mesoderm or ectoderm tissues throughout the culture.

U.S. Patent Application No. 20030180269 describes a composition that comprises stem or progenitor cells from post-partum placenta and umbilical cord blood supplemented with a plurality of embryonic-like stem cells. These cells are stated to be oct-4+ ABC-p+, SSEA3− and SSEA4−. Similarly, U.S. Patent Application No. 20030032179 describes isolated post-partum placenta and cells isolated therefrom, which are stated to exhibit the following phenotype: CD10+, CD29+, CD34−, CD44+, CD45−, CD54+, CD90+, SH2+, SH3+, SH4+, SSEA3−, SSEA4−, OCT-4+ and ABC-p+.

U.S. Patent Application Nos. 20020168763 and 20030027331 describe homozygous stem cells. It is stated that these stem cells are produced from a mitotically activated homozygous post-meiosis I diploid germ cell by fusing two oocytes or two spermatids, preventing the extrusion of the second polar body during oogenesis, allowing the extrusion of the second polar body and spontaneous self-replication under appropriate conditions, or transferring two sperm or two haploid egg nuclei into an enucleated oocyte. This is followed by culturing said activated homozygous post-meiosis I diploid germ cell to form a blastocyst-like mass and isolating homozygous stem cells from the inner cell mass of said blastocyst-like mass.

U.S. Patent Application No. 20020090722 describes a pluripotent cell population, stated to be derived from the method of preparing cytoplast fragments from a mammalian oocyte or fertilized zygote (the cytoplast donor), fusion of a cytoplast fragment with a cell or karyoplast (the nuclear donor) which can be taken from any mammalian species.

U.S. Patent Application No. 20020142457 describes a cell which has been isolated from a living tissue or umbilical blood, and which is stated to be more primitive than hematopoietic or mesenchymal stem cells and to differentiate into all of the three germ layers including the ectoderm, mesoderm and endoderm.

U.S. Patent Application No. 20020164794 describes an unrestricted somatic stem cell (USSC) derived from human umbilical cord blood, placental blood and/or blood samples from newborns. This somatic stem cell is stated to be distinct from but capable of differentiating into mesenchymal stem or progenitor cells, hematopoietic lineage stem or progenitor cells, neural stem or progenitor cells or endothelial stem or progenitor cells.

U.S. Patent Application No. 20030219866 describes dedifferentiated stem cells, or what is stated to be a “stem cell-like cell.”

U.S. Patent Application No. 20030219898 describes mammalian multipotent stem cells (MSCs). These cells can be derived by methods of making more developmentally potent cells from less developmentally potent cells.

U.S. Patent Application No. 20030124720 described what are stated to be pluripotent and germ line competent mammalian stem cells.

U.S. Patent Application No. 20030082803 describes what are stated to be pluripotent or pluripotent-related cells from a mammal, which can be a human, which are produced by modulating activity or expression levels of kinases that alter the cell cycle, such as Cdk2.

U.S. Patent Application Nos. 20020081724 and 20020137204 describes what is stated to be a composition comprising proliferating primate pluripotent stem (pPS) cells, which is essentially free of feeder cells.

U.S. Patent Application No. 20030032177 describes what are stated to be pluripotent or pluripotent-related cells obtained by a method of regulating differentiation potential by manipulating the expression and/or activity of a cell differentiation regulatory molecule in a pluripotent or pluripotent-related cell.

U.S. Patent Application No. 20030087431 describes what is stated to be a stem cell line isolated from composite blastocysts (CBs) that comprise cells derived from non-viable pre-embryos. CBs are produced by dissociation of non-viable pre-embryos into non-nucleated and individual nucleated cells or groups of cells; b) isolation of individual mononucleated cells or groups of mononucleated cells from disaggregated non-viable pre-embryos; c) aggregation of isolated mononucleated cells or groups of mononucleated cells from non-viable pre-embryos in a host zona pellucida; and d) culturing of the zona-encapsulated cell aggregates to allow multiplication and differentiation of cells.

Hematopoietic stem cells for use with methods of the invention can be obtained from pluripotent stem cell sources as well. For example, U.S. Pat. No. 5,914,268 describes a pluripotent cell population for use in the development into hematopoietic cells, progenitors and progeny thereof. The pluripotent cell population is derived by culturing an embryonic stem cell population to obtain an embryoid body cell population, which is then followed by culturing said embryoid body cell population under conditions effective to produce said pluripotent cell population. The culturing conditions comprise an embryonic blast cell medium.

Stem cells of the present invention also include those known in the art that have been identified in organs or tissues (tissue specific stem cells). The best characterized is the hematopoietic stem cell. The hematopoietic stem cell, isolated from bone marrow, blood, cord blood, fetal liver and yolk sac, is the progenitor cell that generates blood cells or following translation reinitiates multiple hematopoietic lineages and can reinitiate hematopoiesis for the life of a recipient. (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827) When transplanted into lethally irradiated animals or humans, hematopoietic stem cells can repopulate the erythroid, neutrophil-macrophage, megakaryocyte and lymphoid hematopoietic cell pool. In vitro, hematopoietic stem cells can be induced to undergo at least some self-renewing cell divisions and can be induced to differentiate to the same lineages observed in vivo.

Stem and/or progenitor cells of the invention preferably comprise a population of cells that have about 50-55%, 55-60%, 60-65% and 65-70% purity (e.g., non-stem and/or non-progenitor cells have been removed or are otherwise absent from the population). More preferably the purity is about 70-75%, 75-80%, 80-85%; and most preferably the purity is about 85-90%, 90-95%, and 95-100%. Purified populations of stem and progenitor cells of the invention can be contacted with a Gαs activating agent before, after or concurrently with purification steps and administered to the subject.

Administered cells of the invention can be autologous (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison.

Various other embodiments are provided, wherein the administered cells of the invention may be genetically altered. In certain embodiments, the blood cells may be transfected with exogenous DNA that encodes, for example, one of the hematopoietic growth factors described elsewhere herein.

Genetic alteration of cells includes all transient and stable changes of the cellular genetic material which are created by the addition of exogenous genetic material. Examples of genetic alterations include any gene therapy procedure, such as introduction of a functional gene to replace a mutated or nonexpressed gene, introduction of a vector that encodes a dominant negative gene product, introduction of a vector engineered to express a ribozyme and introduction of a gene that encodes a therapeutic gene product. Exogenous genetic material includes nucleic acids or oligonucleotides, either natural or synthetic, that are introduced into the stem and progenitor cells. The exogenous genetic material may be a copy of that which is naturally present in the cells, or it may not be naturally found in the cells. It typically is at least a portion of a naturally occurring gene which has been placed under operable control of a promoter in a vector construct.

Various techniques may be employed for introducing nucleic acids into cells. Such techniques include transfection of nucleic acid-CaPO₄ precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid according to the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

In the present invention, the preferred method of introducing exogenous genetic material into cells is by transducing the cells in situ on the matrix using replication-deficient retroviruses. Replication-deficient retroviruses are capable of directing synthesis of all virion proteins, but are incapable of making infectious particles. Accordingly, these genetically altered retroviral vectors have general utility for high-efficiency transduction of genes in cultured cells, and specific utility for use in the method of the present invention. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in the art.

The major advantage of using retroviruses is that the viruses insert efficiently a single copy of the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types. The major disadvantages of using a retrovirus expression vector are (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. Despite these apparent limitations, delivery of a therapeutically effective amount of a therapeutic agent via a retrovirus can be efficacious if the efficiency of transduction is high and/or the number of target cells available for transduction is high.

Yet another viral candidate useful as an expression vector for transformation of cells is the adenovirus, a double-stranded DNA virus. Like the retrovirus, the adenovirus genome is adaptable for use as an expression vector for gene transduction, i.e., by removing the genetic information that controls production of the virus itself. Because the adenovirus functions usually in an extrachromosomal fashion, the recombinant adenovirus does not have the theoretical problem of insertional mutagenesis. On the other hand, adenoviral transformation of a target cell may not result in stable transduction. However, more recently it has been reported that certain adenoviral sequences confer intrachromosomal integration specificity to carrier sequences, and thus result in a stable transduction of the exogenous genetic material.

Thus, as will be apparent to one of ordinary skill in the art, a variety of suitable vectors are available for transferring exogenous genetic material into cells. The selection of an appropriate vector to deliver a therapeutic agent for a particular condition amenable to gene replacement therapy and the optimization of the conditions for insertion of the selected expression vector into the cell, are within the scope of one of ordinary skill in the art without the need for undue experimentation. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any nontranslated DNA sequence which works contiguous with the coding sequence (in cis) to change the basal transcription level dictated by the promoter. Preferably, the exogenous genetic material is introduced into the cell genome immediately downstream from the promoter so that the promoter and coding sequence are operatively linked so as to permit transcription of the coding sequence. A preferred retroviral expression vector includes an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., 1991, Proc. Natl. Acad. Sci. USA, 88:4626-4630), adenosine deaminase, phosphoglycerol kinase (PGK), pyruvate kinase, phosphoglycerol mutase, the actin promoter (Lai et al., 1989, Proc. Natl. Acad. Sci. USA, 86:10006-10010), and other constitutive promoters known to those of skill in the art. In addition, many viral promoters function constitutively in eukaryotic cells. These include: the early and late promoters of SV40; the long terminal repeats (LTRS) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector preferably includes a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene and/or signal sequence (described below) is deemed to be within the scope of one of ordinary skill in the art without undue experimentation.

The selection and optimization of a particular expression vector for expressing a specific gene product in an isolated cell is accomplished by obtaining the gene, preferably with one or more appropriate control regions (e.g., promoter, insertion sequence); preparing a vector construct comprising the vector into which is inserted the gene; transfecting or transducing cultured cells in vitro with the vector construct; and determining whether the gene product is present in the cultured cells.

It also is possible to take the increased numbers of stem and progenitor cells produced according to the invention and stimulate them with hematopoietic growth agents that promote hematopoietic cell maintenance, expansion and/or differentiation, and also influence cell localization, to yield the more mature blood cells, in vitro. Such expanded populations of blood cells may be applied in vivo as described above, or may be used experimentally as will be recognized by those of ordinary skill in the art. Such differentiated cells include those described above, as well as T cells, plasma cells, erythrocytes, megakaryocytes, basophils, polymorphonuclear leukocytes, monocytes, macrophages, eosinophils and platelets.

In all of the culturing methods according to the invention, except as otherwise provided, the media used is that which is conventional for culturing blood cells. Examples include RPMI, DMEM, Iscove's, etc. Typically these media are supplemented with human or animal plasma or serum. Such plasma or serum can contain small amounts of hematopoietic growth factors. The media used according to the present invention, however, can depart from that used conventionally in the prior art. Methods of culturing stem and progenitor cells may be specific to the particular type of cell isolated or generated as eth case may be, and are generally well described in the art.

The growth agents of particular interest in connection with the present invention are hematopoietic growth factors. By hematopoietic growth factors, it is meant factors that influence the survival, proliferation or differentiation of hematopoietic stem and progenitor cells. Growth agents that affect only survival and proliferation, but are not believed to promote differentiation, include the interleukins 3, 6 and 11, stem cell factor and FLT-3 ligand. Hematopoietic growth factors that promote differentiation include the colony stimulating factors such as GMCSF, GCSF, MCSF, Tpo, Epo, Oncostatin M, and interleukins other than IL-3, 6 and 11. The foregoing factors are well known to those of ordinary skill in the art. Most are commercially available. They can be obtained by purification, by recombinant methodologies or can be derived or synthesized synthetically.

Stromal cell conditioned medium refers to medium in which lymphoreticular stromal cells have been incubated. The incubation is performed for a period sufficient to allow the stromal cells to secrete factors into the medium. Such stromal cell conditioned medium can then be used to supplement the culture of hematopoietic stem and progenitor cells promoting their proliferation and/or differentiation.

Thus, when cells are cultured without any of the foregoing agents, it is meant herein that the cells are cultured without the addition of such agent except as may be present in serum, ordinary nutritive media or within the blood product isolate, unfractionated or fractionated, which contains the hematopoietic stem and progenitor cells.

Current practice during bone marrow transplantation involves the isolation of bone marrow cells from the bone marrow and/or peripheral blood of donor subjects. One of skill in the art would be aware of methods for isolating blood cells from peripheral blood. For example blood in PBS is loaded into a tube of Ficoll (Ficoll-Paque, Amersham) and centrifuged at 1500 rpm for 25-30 minutes. After centrifugation the white center ring is collected as containing hematopoietic stem cells.

About one third of these subjects do not “yield” enough hematopoietic progenitor cells from their bone marrow and/or peripheral blood so that their marrow can be considered suitable for transplantation. Using the methods of the invention, the “yield” may be enhanced. For example, agents that activate Gαs result in “mobilization” of administered cells (e.g., blood cells, stem and progenitor cells, hematopoietic stem and progenitor cells) and the efficiency of a harvested population of blood cells, or stem and/or progenitor cells, may be improved. This then results in an increase in the number of donor samples that may be used in transplantation or a decrease in the size of the donor samples that is required.

Accordingly, cells of the invention can be contacted with a Gαs activating agent prior to or concurrently with transplantation of the cells into a subject. The Gαs activating agent includes any agent capable of activating Gαs or variants of Gαs, such as pertussis toxin, or variants of pertussis toxin.

Preferably, the cells are washed or otherwise purified of the Gαs activating agent prior to administration to the subject. Cells can be washed, for example, in phosphate buffered saline.

Methods of the invention are useful as a supplemental treatment to chemotherapy, e.g., blood cells may be isolated from a subject that will undergo chemotherapy, and after the therapy the cells can be returned (e.g. ex vivo Gαs activation can be performed on the isolated cells according to methods of the invention). Thus, the subject in some embodiments is a subject undergoing or expecting to undergo an immune cell depleting treatment such as chemotherapy. Most chemotherapy agents used act by killing all cells going through cell division. Bone marrow is one of the most prolific tissues in the body and is therefore often the organ that is initially damaged by chemotherapy drugs. The result is that blood cell production is rapidly destroyed during chemotherapy treatment. Methods of the invention can improve the recovery of the bone marrow by increasing the return and engraftment of regenerative stem and progenitor cells.

In specific embodiments, hematopoietic stem and progenitor cells are mobilized from the bone marrow to the peripheral blood and blood samples are isolated in order to obtain the hematopoietic stem and progenitor cells. These cells can be treated with Gαs activating agents and transplanted immediately or they can be further processed in vitro, either prior to, following or concurrently with Gαs activation. For instance, the cells can be expanded in vitro and/or they can be subjected to an isolation or enrichment procedure. It will be apparent to those of ordinary skill in the art that the crude or unfractionated blood products can be enriched for cells having hematopoietic stem or progenitor cell characteristics. Some of the ways to enrich include, e.g., depleting the blood product from the more differentiated progeny. Methods for isolation of blood cells are well-known in the art, and typically involve purification techniques based on cell surface markers and functional characteristics. The more mature, differentiated cells can be selected against, via cell surface molecules they express. Additionally, the blood product can be fractionated selecting for CD34⁺ cells. Such selection can be accomplished using, for example, commercially available magnetic anti-CD34 beads (Dynal, Lake Success, N.Y.).

Methods of the invention further provides methods of treating a disorder or disease. As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

The methods of the invention can be used to treat any disease or disorder in which it is desirable to increase the amount of hematopoietic stem and progenitor cells in the bone marrow or mobilize hematopoietic stem and progenitor cells to the bone marrow. For example, methods of the invention can be used to treat patients requiring a bone marrow transplant or a hematopoietic stem or progenitor cell transplant, such as cancer patients undergoing chemo and/or radiation therapy. Methods of the present invention are particularly useful in the treatment of patients undergoing chemotherapy or radiation therapy for cancer, including patients suffering from myeloma, non-Hodgkin's lymphoma, Hodgkins lyphoma, leukaemia, and solid tumors (breast cancer, ovarian cancer, brain cancer, prostate cancer, lung cancer, colon cancer, skin cancer, liver cancer, or pancreatic cancer). Methods of the present invention can also be used in the treatment of patients suffering from aplastic anemia, an immune disorder (severe combined immune deficiency syndrome or lupus), myelodysplasia, thalassemaia, sickle-cell disease or Wiskott-Aldrich syndrome.

Disorders treated by methods of the invention can be the result of an undesired side effect or complication of another primary treatment, such as radiation therapy, chemotherapy, or treatment with a bone marrow suppressive drug, such as zidovadine, chloramphenical or gangciclovir. Such disorders include neutropenias, anemias, thrombocytopenia, and immune dysfunction. In addition, methods of the invention can be used to treat damage to the bone marrow caused by unintentional exposure to toxic agents or radiation.

The disorder to be treated can also be the result of an infection (e.g., viral infection, bacterial infection or fungal infection ) causing damage to stem or progenitor cells of the bone marrow.

In addition to the above, further conditions which can benefit from treatment using methods of the invention include, but are not limited to, lymphocytopenia, lymphorrhea, lymphostasis, erythrocytopenia, erthrodegenerative disorders, erythroblastopenia, leukoerythroblastosis; erythroclasis, thalassemia, myelofibrosis, thrombocytopenia, disseminated intravascular coagulation (DIC), immune (autoimmune) thrombocytopenic purpura (ITP), HIV inducted ITP, myelodysplasia; thrombocytotic disease, thrombocytosis, congenital neutropenias (such as Kostmann's syndrome and Schwachman-Diamond syndrome), neoplastic associated—neutropenias, childhood and adult cyclic neutropaenia; post-infective neutropaenia; myelo-dysplastic syndrome; and neutropaenia associated with chemotherapy and radiotherapy.

In other embodiments, the invention provides methods for identifying agents capable of increasing stem cell engraftment comprising: providing a first portion of cells which express Gαs; providing a second portion of cells which do not express Gαs; contacting the first and second portions of cells with a test compound; and detecting cAMP expression in both the first and second portions of cells, wherein a test compound which increases cAMP expression from the first portion of cells but not the second portion of cells is identified as an agent capable of increasing stem cell engraftment. In a specific embodiment, the cells are bone marrow mononuclear cells or bone marrow lin⁻ cells. Assays can be carried out in the culture systems previously described.

The methods may further comprise additional steps, such as providing a third portion of cells which are hematopoietic progentor cells and a fourth portion of cells which are hematopoietic progenitor cells; contacting the third portion of cells with the test compound identified as an agent capable of increasing stem cell engraftment; administering the third portion of cells to a test subject; administering the fourth portion of cells to a test subject; and detecting engraftment of the third and fourth portion of cells in the bone marrow of the test subjects, wherein a test compound which increases the number of engrafted cells from the third portion of cells, as compared to the fourth portion of cells, is confirmed as agent capable of increasing stem cell engraftment. In a preferred embodiment, the third and fourth portions of cells are bone marrow lin⁻ cells. In a further preferred embodiment, the subject is a mouse. In still a further preferred embodiment, steps (h) and (j) are performed eight weeks apart.

This invention is further illustrated by the following examples which should not be construed as limiting. The contents of all references, patents and published patent applications cited throughout this application, as well as the sequence listing and the figures, are incorporated herein by reference.

### EXAMPLES

### Example 1

### Gαs^{-/-} Cells Do Not Translocate to the Bone Marrow

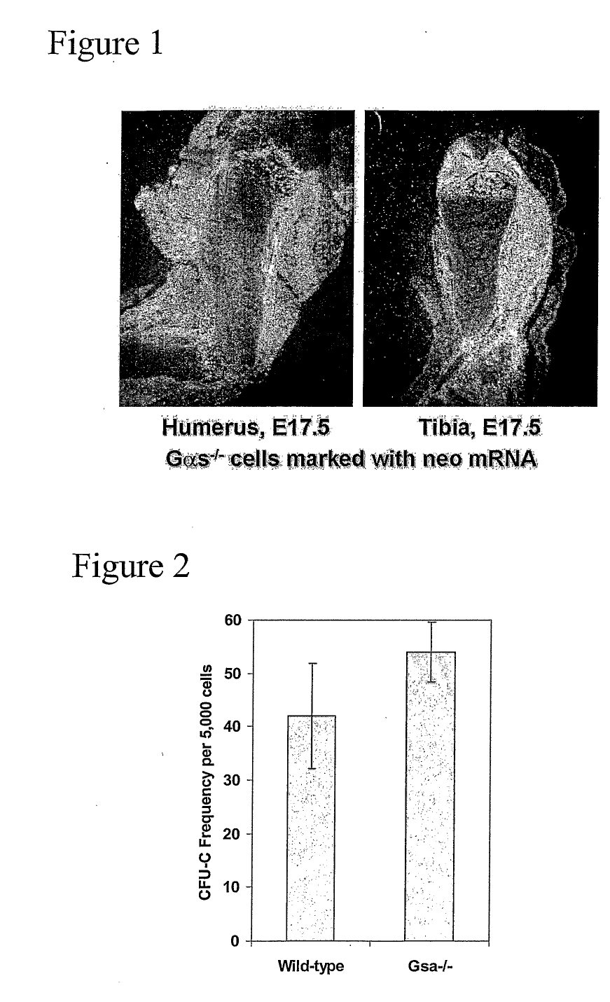

A chimeric mouse model was generated using wild-type blastocysts and ES cells that were Gαs^{-/-} due to a homozygous knockout of the GNAS1 gene. Fetal mice revealed chimerism in all tissues examined, except for the bone marrow where no contribution from the Gαs^{-/-} cells was seen (FIG. 1). This effect was likely a result of the inability of the knockout cells to home or engraft in the bone marrow environment. These results demonstrate that hematopoietic stem cell (“HSC”) homing and engraftment to the bone marrow environment either during development or following transplantation is dependent upon Gαs-coupled receptors.

### Example 2

### Gαs^{-/-} Cells Can Differentiate Into Hematopoietic Cells

To rule out the possibility that the lack of Gαs^{-/-} cells in the bone marrow of the chimeric mice was due to an inability of the knockout cells to differentiate into cells of the hematopoietic lineages, in vitro hematopoietic cell assays were performed. Hematopoietic cells were purified from embryonic day 17 fetal liver by FACS based on their expression of only CD45.2 (Gαs^{-/-}) or CD45.1 and CD45.2 (wild-type). Colony forming-unit assays, which measure the progenitor activity, demonstrated no differences in the ability of the cells to form hematopoietic colonies (FIG. 2).

### Example 3

### Pharmacological Inhibition of Gαs in Adult Cells Does Not Inhibit Stem Cell Differentiation

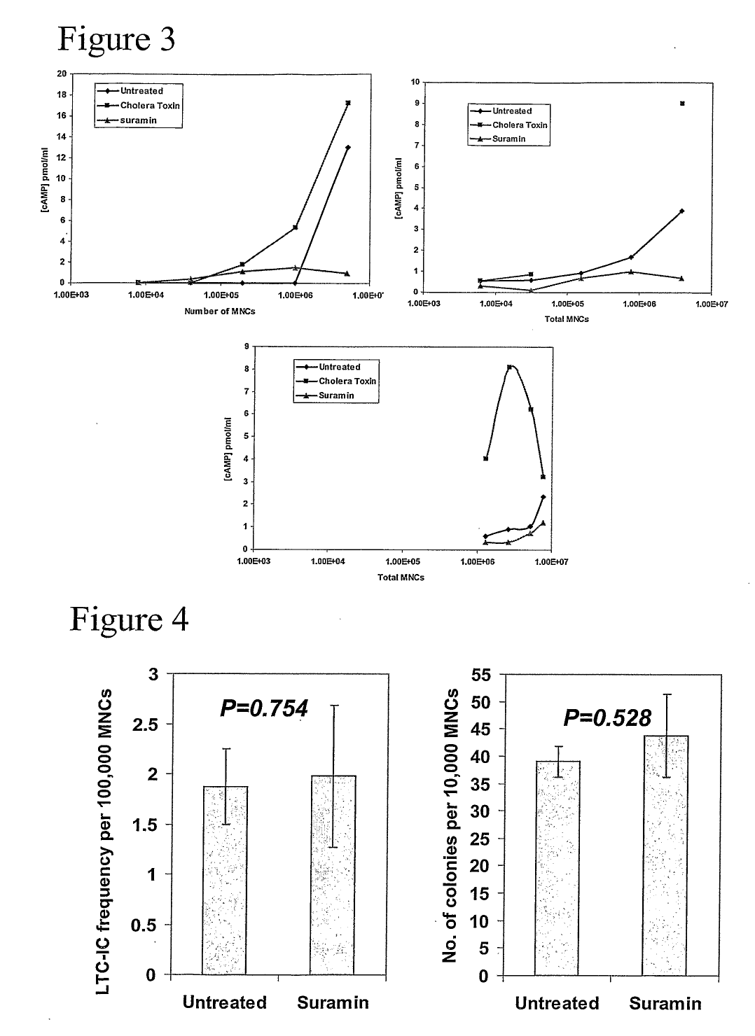

The role of Gαs-coupled receptors in directing hematopoietic stem cells to the bone marrow was studied in the chimeric mouse model of Example 1. The role of Gαs-coupled receptors in directing adult hematopoietic stem cells to the adult bone marrow was assessed following injection of adult hematopoietic stem cells into the peripheral circulation. Adult hematopoietic stem cells were treated with suramin, an inhibitor of Gαs activity prior to administration. Treatment of bone marrow mononuclear cells with suramin resulted in a reduction in the cAMP production in these cells, which is indicative of an inhibition of Gαs signaling (FIG. 3). However, suramin treatment did not result in any impairment of the ability of the cells to perform in both CFU-C and LTC-IC assays (FIG. 4) demonstrating that it did not have any myelotoxicity.

### Example 4

### Suramin Treatment Inhibits In Vivo Homing of Hematopoietic Stem Cells to the Bone Marrow

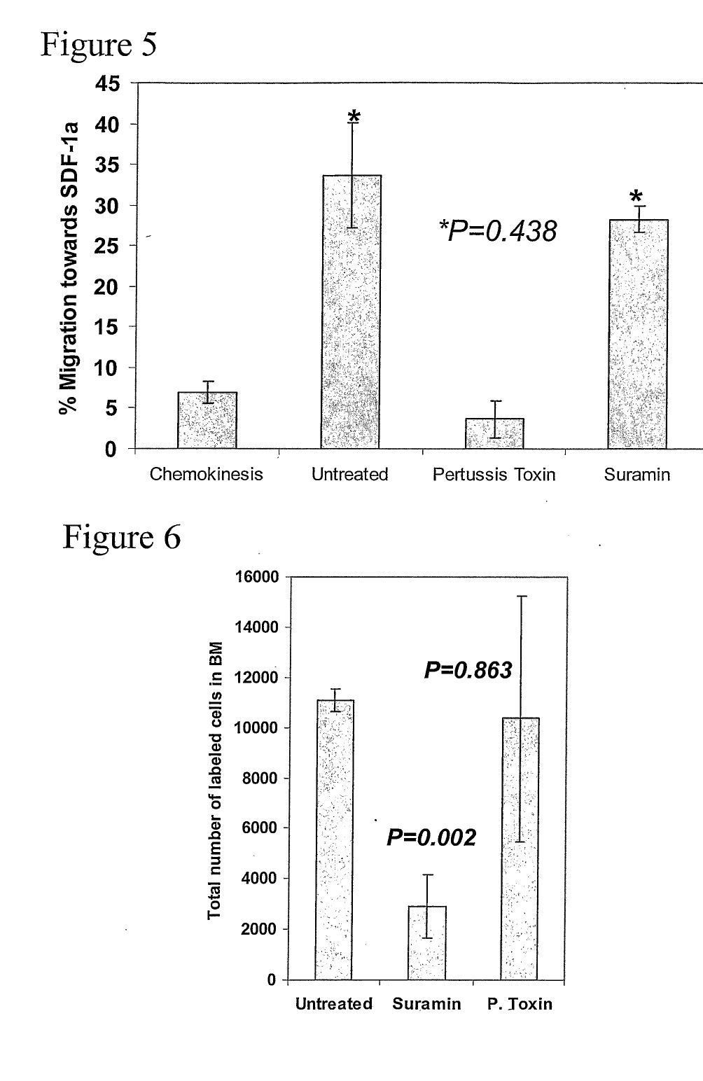

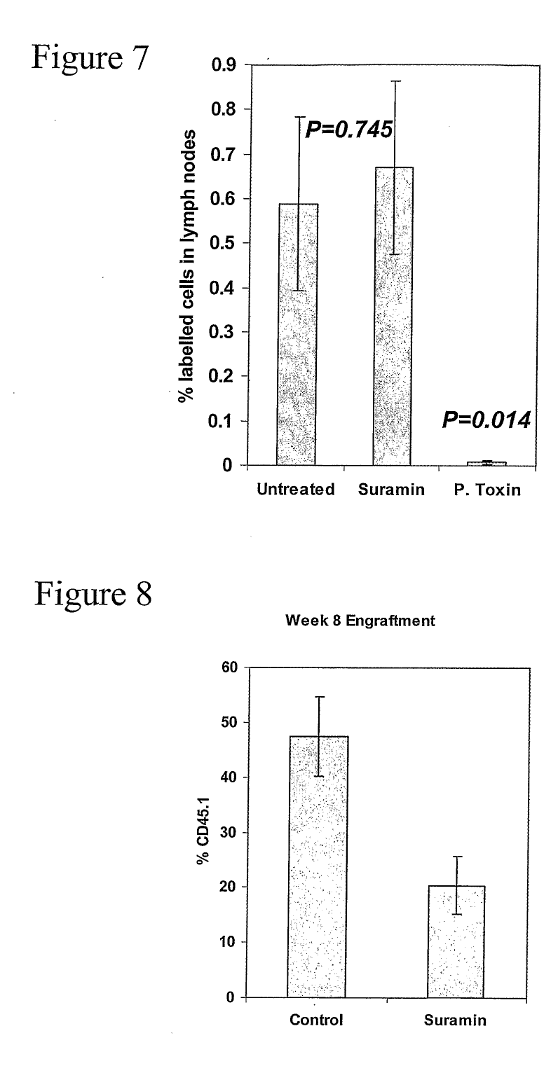

At present, transmigration of hematopoietic stem cells towards SDF-1α across a membrane is used as an in vitro model for stem cell homing in vivo. Therefore, transmigration in response to SDF-1α was examined in stem cells that had been pretreated with suramin. Suramin treatment had no effect on the transmigration of bone marrow lin⁻ cells, whereas treatment of the same cells with pertussis toxin, another inhibitor of Gαs activity, led to a decrease in transmigration similar to that observed for the chemokinesis controls (FIG. 5). On the other hand, suramin treatment of bone marrow lin⁻ cells did result in an impairment of the cells to home to the bone marrow, whereas treatment of the same cells with pertussis toxin did not (FIG. 6). This effect on the homing of the cells was specific to lin⁻ homing to the bone marrow, as treatment of lymph node lymphocytes with suramin had no effect on their homing to the lymph nodes. In these experiments however, pertussis toxin did almost completely abolish lymphocyte homing (FIG. 7).

To assess whether the reduction in the level of homing caused by the treatment of the cells with suramin also caused a reduction in the level of engraftment, competitive repopulation assays were performed utilizing CD45.1 and CD45.2 mice. In these experiments CD45.1 cells were either treated with suramin or untreated. They were then injected with an equal number of CD45.2 whole BM MNCs into lethally irradiated CD45.2 mice. Eight weeks following injection the contribution to bone marrow hematopoiesis of the untreated or treated cells was analyzed by flow cytometry. Treatment of bone marrow MNCs led to a reduction in their ability to engraft and establish hematopoiesis in the bone marrow (FIG. 8) due to a lack of ability of the cells to home to the bone marrow environment.

### Example 5

### Stimulation of Gαs Activity Results in Increased Homing of Hematopoietic Stem Cells to the Bone Marrow

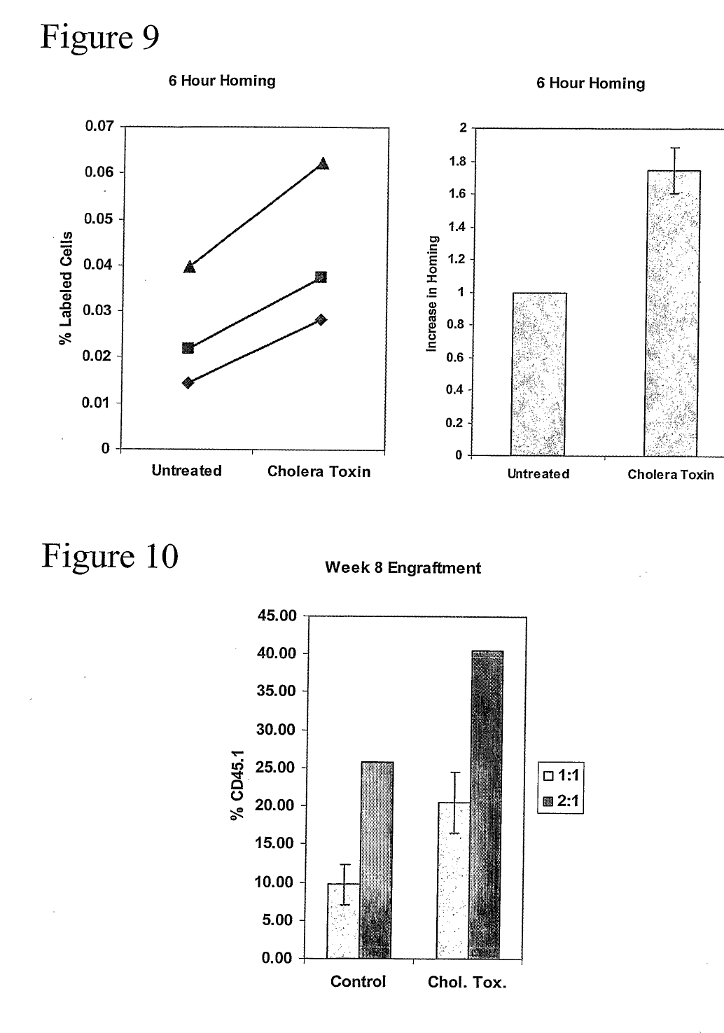

Whether pharmacologic stimulation of Gαs signaling with cholera toxin could result in enhanced homing and engraftment of hematopoietic stem cells in the bone marrow was tested. Treatment of hematopoietic progenitor cells with cholera toxin (1 hour), an activator of Gαs, followed by injection into the peripheral circulation of lethally irradiated mice led to an approximate 75% increase in the number of cells homing to the bone marrow (FIG. 9). Similarly, analysis of the engraftment potential of cells treated with cholera toxin demonstrated an enhanced contribution in competitive transplants by cells treated with cholera toxin (FIG. 10).

### Example 6

### Conditional knockout of the Gsα gene Does Not Affect Differentiation of Hematopoietic Stem Cells

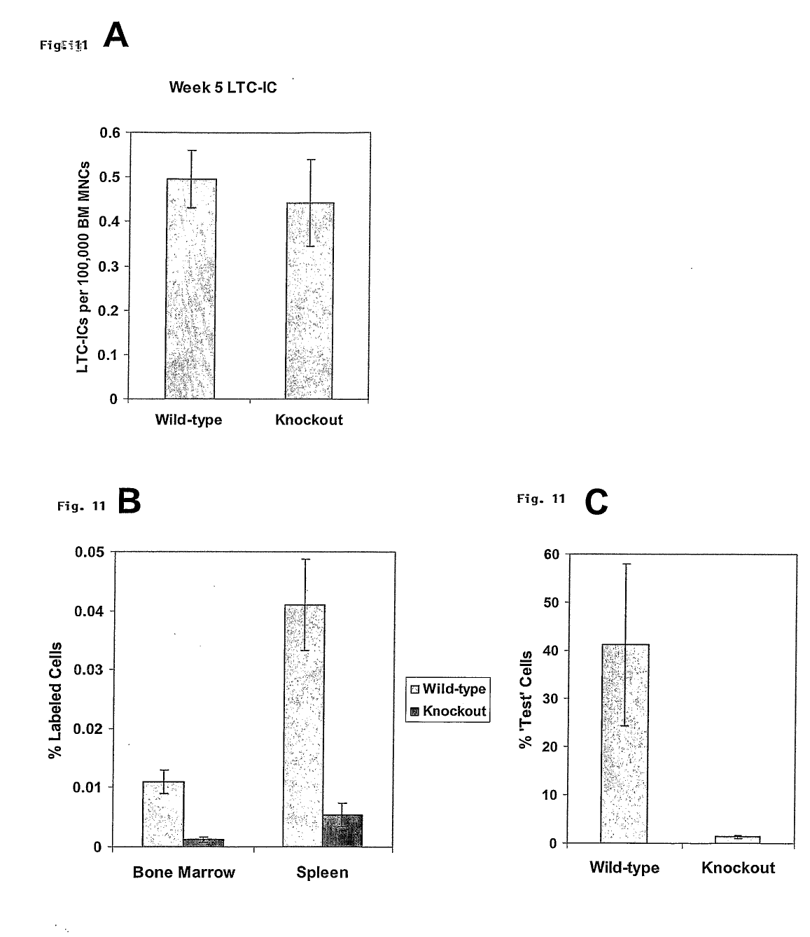

A conditional knockout of the Gₛα gene in mice was created and the effects of the knock out were analyzed on the hematopoietic stem cell population. Specifically, mice that had a ‘floxed’ Gₛα allele (Gₛα^{fl/fl}) (Sakamoto et al. J. Bone Miner. Res. 20(4): 663-71, 2005) were bred with mice that had Cre recombinase under the control of the Mx1 promoter (Mx1-Cre) to create Gₛα^{fl/fl}Mx1-Cre mice. Mx1-Cre mice are commercially available from Jackson Laboratory. In these mice deletion of the Gₛα gene was induced by three injections of polyI.polyC over a five-day period. At the end of this period, the mice were sacrificed and the mononuclear cells were obtained from the bone marrow. The ability of the cells to perform in in vitro assays of hematopoietic stem cell function was analyzed. As shown in FIG. 11A, bone marrow mononuclear cells obtained from mice with the conditional knockout of Gₛα, or Gₛα^{+/+}Mx1-Cre mice (wild-type) that were treated under identical conditions demonstrated no difference in their LTC-IC frequency. These results suggest that deletion of the Gₛα gene has no effect on the ability of the hematopoietic stem cell to differentiate into fully mature hematopoietic cells.

The ability of primitive hematopoietic cells to localize to the bone marrow and spleen microenvironment was assessed in vivo. Bone marrow mononuclear cells obtained from the ‘wild-type’ or ‘knockout’ mice were fractionated to isolate primitive lineage-negative cells. The cells were labeled with a fluorescent dye and injected into irradiated wild-type hosts. Six hours following injection of the cells, the number of cells localizing to the bone marrow and spleen were calculated by flow cytometry. As demonstrated in FIG. 11B, the Gₛα knockout cells had a significant impairment in the ability to localize to the hematopoietic tissues of the bone marrow and spleen.

Finally, the in vivo bone marrow reconstituting ability of wild-type versus knockout cells was examined. Mononuclear cells from the conditional knockout mouse or wild-type mouse were mixed with an equal number of ‘competitor’ wild-type cells. These cells were then injected into lethally irradiated wild-type mice. Eight weeks following injection of the cells, the relative contribution from the different cell sources was calculated by flow cytometry. As demonstrated in FIG. 11C, the knockout cells had a significant impairment in the ability to engraft in the bone marrow in vivo.

In summary, using a mouse model in which a conditional knockout of the Gₛα gene was made, these data demonstrate that the lack of Gₛα does not affect the hematopoietic differentiation capabilities of hematopoietic stem cells. The ability of the cells to home, and thus engraft, in the bone marrow microenvironment is severely impaired, highlighting that Gₛα is a key component of this process.

Those skilled in the art will recognize, or be able to ascertain using no more than routine experimentation, many equivalents to the specific embodiments of the invention described herein. Such equivalents are intended to be encompassed by the following claims.

1. A method for increasing stem cell engraftment in a subject in need thereof, the method comprising: contacting blood cells to be transplanted into the subject with a Gas activating agent and administering the cells to the subject, thereby increasing stem cell engraftment in the subject.

2. The method of claim 1, further comprising substantially removing the Gas activating agent from the cells prior to administering the cells to the subject.

3. The method of claim 1, wherein substantially removing the Gas activating agent comprises washing the cells.

4. The method of claim 1, further comprising treating the subject with an amount of radiation or chemotherapy sufficient to ablate the subject's bone marrow prior to administration of the cells.

5. The method of claim 1, wherein the blood cells comprise bone marrow cells.

6. The method of claim 1, wherein the blood cells comprise hematopoietic stem or progenitor cells.

7. The method of claim 1, wherein the blood cells comprise umbilical cord blood.

8. The method of claim 1, wherein the Gas activating agent comprises cholera toxin.

9. The method of claim 1, wherein the subject is a human.

10. The method of claim 1, wherein the subject is suffering from a disorder selected from the group consisting of leukemia, aplastic anemia, lymphoma, multiple myeloma, an immune disorder, myelodysplasia, thalassemaia, and sickle-cell disease, Wiskott-Aldrich syndrome, and solid tumors.

11. The method of claim 10, wherein the lymphoma is selected from the group consisting of Hodgkin's disease and Non-Hodgkin's lymphoma.

12. The method of claim 10, wherein the immune disorder is selected from the group consisting of severe combined immune deficiency syndrome and lupus.

13. The method of claim 10, wherein the solid tumor is selected from the group consisting of breast cancer, ovarian cancer, brain cancer, prostate cancer, lung cancer, colon cancer, skin cancer, liver cancer, and pancreatic cancer.

14. (canceled)

15. A method for increasing stem cell mobilization to the bone marrow of a subject in need thereof, the method comprising: contacting blood cells to be transplanted into the subject with a Gas activating agent and administering the cells to the subject, thereby increasing stem cell mobilization to the bone marrow of the subject.

16. The method of claim 15, further comprising substantially removing the Gas activating agent from the cells prior to administering the cells to the subject.

17. The method of claim 15, wherein substantially removing the Gas activating agent comprises washing the cells.

18. The method of claim 15, further comprising treating the subject with an amount of radiation or chemotherapy sufficient to ablate the subject's bone marrow prior to administration of the cells.

19. The method of claim 15, wherein the blood cells comprise bone marrow cells.

20. The method of claim 15, wherein the blood cells comprise hematopoietic stem or progenitor cells.

21. The method of claim 15, wherein the blood cells comprise umbilical cord blood.

22. The method of claim 15, wherein the Gas activating agent comprises cholera toxin.

23. The method of claim 15, wherein the subject is a human.

24. The method of claim 15, wherein the subject is suffering from a disorder selected from the group consisting of leukemia, aplastic anemia, lymphoma, multiple myeloma, an immune disorder, myelodysplasia, thalassemaia, and sickle-cell disease, Wiskott-Aldrich syndrome, and solid tumors.

25. The method of claim 24, wherein the lymphoma is selected from the group consisting of Hodgkin's disease and Non-Hodgkin's lymphoma.

26. The method of claim 24, wherein the immune disorder is selected from the group consisting of severe combined immune deficiency syndrome and lupus.

27. The method of claim 24, wherein the solid tumor is selected from the group consisting of breast cancer, ovarian cancer, brain cancer, prostate cancer, lung cancer, colon cancer, skin cancer, liver cancer, and pancreatic cancer.

28. The method according to claim 1, further comprising obtaining the Gas activating agent.

29-36. (canceled)
